(19) United States
(12) Patent Application Publication
(10) Pub. No.: US 2010/0243495 A1
(43) Pub. Date: Sep. 30, 2010

(54) CONTAINER FOR WASTE DISPOSAL

(75) Inventor: Alan Julian Segal, Cheshire (GB)

Correspondence Address: GREENBLUM & BERNSTEIN, P.L.C., 1950 ROLAND CLARKE PLACE, RESTON, VA, 20191, US

(73) Assignee: ASTEK INNOVATIONS LIMITED, Cheshire (GB)

(21) Appl. No.: 12/523,433

(22) PCT Filed: Jan. 17, 2008
(86) PCT No.: PCT/GB2008/000169

§ 371 Date: Oct. 16, 2009

(30) Foreign Application Priority Data
Jan. 18, 2007 (GB) 0701131.5

(51) Int. Cl. B65D 83/24; A61B 19/02

(52) U.S. Cl. 206/366; 206/370

(57) ABSTRACT

A container, for disposal of unsafe waste such as so-called “sharps” waste or other contaminated surgical, medical, or other materials, is in the form of a receptacle with an inlet for waste. In one embodiment, the inlet has gripper fingers so that a used needle can be pulled off a syringe, to fall into the receptacle, after insertion of the needle through the gripper fingers and then withdrawing the syringe away from the fingers. A base is provided for attaching the receptacle to a surface. The receptacle is interconnected with the base through a channel on the base, and a projecting part on the receptacle. The part can slide along the channel in one direction for interconnection of the receptacle and the base. A retention device is provided to prevent sliding in the opposite longitudinal direction e.g. when the syringe is withdrawn in such direction away from the fingers.

This invention relates to a container for disposal of unsafe waste such as so-called ‘sharps’ waste or other contaminated surgical or medical implements or materials.

WO 03/047664A describes a sharps container which can be used for disposal of dental syringe needles. The container comprises a generally rectangular receptacle which has an inlet in the form of a receiving tube extending along a top side. A used syringe is inserted into the tube to engage a gripping mechanism whereby withdrawal of the syringe then causes the needle to be detached and fall into the receptacle.

This provides an efficient and safe means of detaching the used syringe, but there is the problem of safely holding the receptacle in position particularly during the detachment procedure. Manual gripping of the receptacle may not be desirable having regard to the danger of needle stick injuries.

U.S. Pat. No. 5,058,764 describes a hospital sharps container with a wall-mounting bracket. The container comprises a generally rectangular receptacle with a covered opening at its top side and the bracket comprises an upright plate which is shaped to hook over an upper conformation of the receptacle and has a spring loaded latch at its lower end which engages a recess in the bottom of the receptacle. A key is provided for retracting the latch to allow removal of the receptacle.

This arrangement is specific to wall mounting and relies on a relatively complicated interfit construction for both the receptacle and the bracket. Accordingly it lacks versatility and convenience of use and it may be difficult to manufacture inexpensively in requisite reliable and robust form.

It is also known to provide a sharps container with a generally square receptacle and an integral circular projecting structure beneath its bottom side which clips into a circular receiving aperture in a separate square base. The base can be fixed to a surface with self adhesive tabs. The projecting structure is moved into and out of locking engagement with the base by rotation of the receptacle.

This arrangement requires the base always to be attached to the receptacle for stability and the rotational interconnection may be difficult to manufacture inexpensively in requisite reliable and robust form.

An object of the present invention is to provide a container with a mounting device for disposal of unsafe waste which is versatile and convenient to use and which can be readily and inexpensively manufactured in robust and reliable form.

According to the invention therefore there is provided a container for disposal of unsafe waste comprising a receptacle with an inlet thereto for waste and a mounting device for the receptacle in the form of a base which can be attached to a surface, the receptacle being interconnectable with the base through cooperable configurations characterised in that the cooperable configurations comprise respectively a channel and a projecting part, said channel and part being interconnectable by longitudinal sliding of the part along the channel, and at least one retention device being provided to restrict the extent of said sliding in at least one longitudinal direction.

With this arrangement, the use of a sliding interconnection between the receptacle and base enables a robust, reliable construction to be readily and inexpensively manufactured. On the one hand, the base can be readily adapted for attachment to any suitable surface whether a horizontal table top or a vertical wall, so as to ensure stable support of the receptacle relative to such surface. On the other hand the longitudinally sliding structures can readily be formed so as to enable the receptacle to stand stably on a horizontal table top with or without the base. The container of the invention is therefore convenient and versatile in use. Moreover, the retention device can reliably ensure that the receptacle does not easily detach from the base when a force is applied to the receptacle in at least one longitudinal direction e.g. on insertion of waste such as a syringe needle into the receptacle inlet and/or on withdrawal of an associated implement, such as the syringe barrel, from the inlet.

The container of the invention may be used for any suitable purpose. Thus, it may be used for sharps e.g. syringe needles, syringes with needles, scalpel blades, etc., or contaminated waste such as syringe cartridges, surgical dressings, bandages, gauzes, sponges and the like.

In one embodiment the container is used with syringes such as dental syringes of the kind having a needle which after use can be pulled off for disposal. Thus, the inlet of the receptacle of the container may have a gripper or abutment device for engagement with a pull off hub of the needle or to a pull off mounting device on which the needle is mounted so that the needle can be inserted into the inlet on the syringe and retraction of the syringe causes the needle to be pulled off by engagement of the needle hub or mounting device with the gripper or abutment device.

In particular, a gripper arrangement of the kind described in WO 03/047664A i.e. having deflectable gripper fingers at the leading end of a tubular syringe-receiving inlet may be provided.

However, it is to be understood that the invention is not restricted to use with such a gripper arrangement, nor even to use with pull-off needles, and the invention may find application in the context of different needle pull-off gripper or abutment devices and also in the context of disposal of waste other then needles or syringes. Accordingly the inlet may take any suitable form and thus may comprise a tubular inlet as mentioned above or may comprise any other suitable structure such as a keyhole slot or a covered opening, preferably a tortuous opening to prevent easy return of waste, particularly with a lid or other openable cover, or any combination of these.

With regard to the receptacle, this is preferably of the nature of a bin or box having closed or closeable side walls, a bottom wall and a top wall. The inlet may be in the top wall and the top wall may be defined by a separate lid structure. The body of the receptacle defined by the side and bottom walls may be of any suitable shape e.g. of generally square or rectangular cross-section whereby the bottom wall may correspondingly be square or rectangular. In one embodiment the cross-section of the body of the receptacle is elongate rectangular with its long axis in the aforesaid longitudinal direction of the said channel.

With regard to the aforesaid base this may be of any suitable structure. In one embodiment it is of larger cross-section, preferably in all transverse directions, than the receptacle. The base may have a flat underside or at least coplanar parts for positioning on a level support surface. Alternatively or additionally the base may be provided with a bracket or extension e.g. an upwardly directed side extension for wall mounting. Other mounting arrangements are also possible.

Fixing of the base relative to a surface may be achieved by means of one or more adhesive tabs between the base and the surface. Alternatively or additionally screw fixings or the like may be used.

With regard to the interconnectable projecting part and channel, these may be provided in any suitable configuration. In one embodiment the channel is provided in the base and the projecting part on the receptacle although the opposite arrangement may also be used.

Preferably the arrangement is such that the underside of the receptacle is flat or has coplanar areas so that is can stand stably on a level surface without the base.

Especially where the receptacle is of elongate rectangular cross-section, the projecting part, when provided on the receptacle, may be defined by a lowermost portion of the receptacle.

The channel and the projecting part may be of any suitable length and cross-section. In one embodiment they are substantially equal in length. Preferably the configuration on the receptacle e.g. the projecting part, extends along substantially all of the corresponding dimension (e.g. length) of the receptacle, and the configuration on the base e.g. the channel is at least substantially as long as the configuration on the receptacle.

With regard to the said retention device this may provide permanent retention of the receptacle and the base. Alternatively it may be releasable so that the receptacle can be detached from the base when desired.

The said retention device may be operable to restrict the extent of sliding along the channel in both directions. Thus, in one embodiment, the device comprises a closure or abutment at one end of the channel and a catch member at the opposite end, the catch member being spring urged or otherwise arranged so as to deflect to allow sliding of the projecting part passed the catch member into the channel and then to return to a locking position at which it obstructs return sliding of the projecting part. The catch member may be further manually deflectable to allow return sliding of the projecting part for detachment of the projecting part from the base.

A further permanent or releasable retention arrangement may be provided to restrain separation of the projecting part out of the channel transversely to the sliding direction. This may comprise a longitudinal guide arrangement such as one or more side projections or ribs on the projecting part engageable with one or more side projections or ribs or grooves on the channel or vice versa.

The invention will now be described further by way of example only and with reference to the accompanying drawings in which:

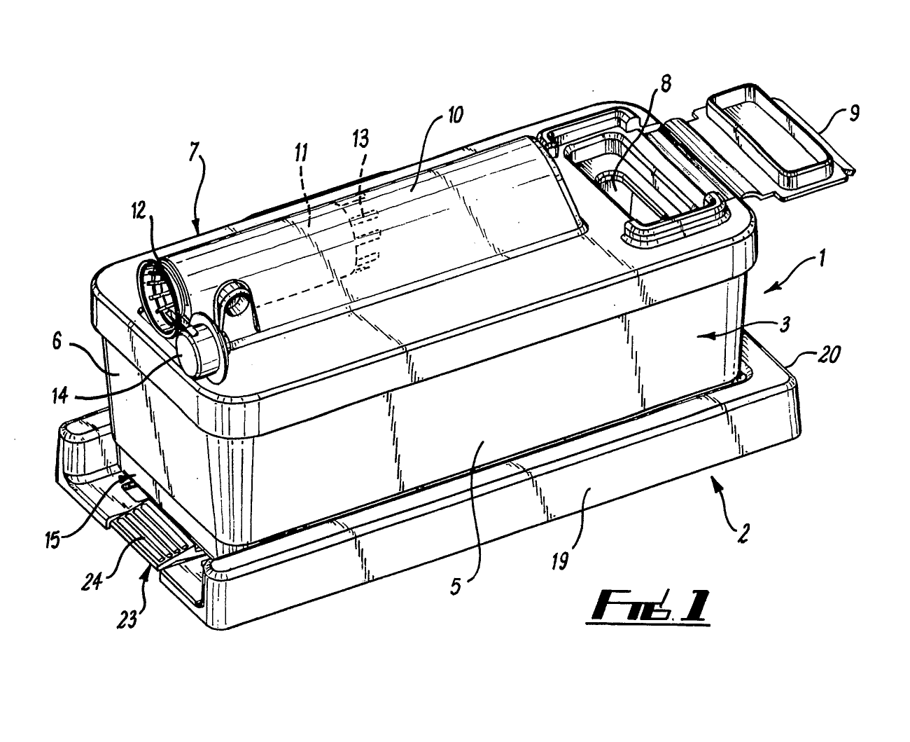

FIG. 1 is a top perspective view of one form of an assembled container according to the invention.

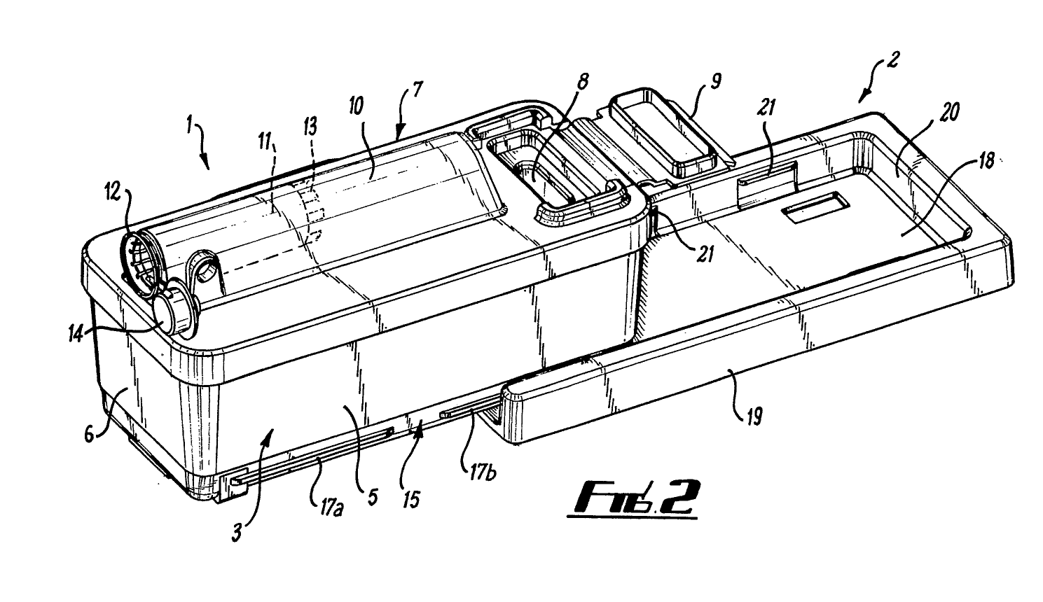

FIG. 2 is a similar view to FIG. 1 in partially disassembled foam;

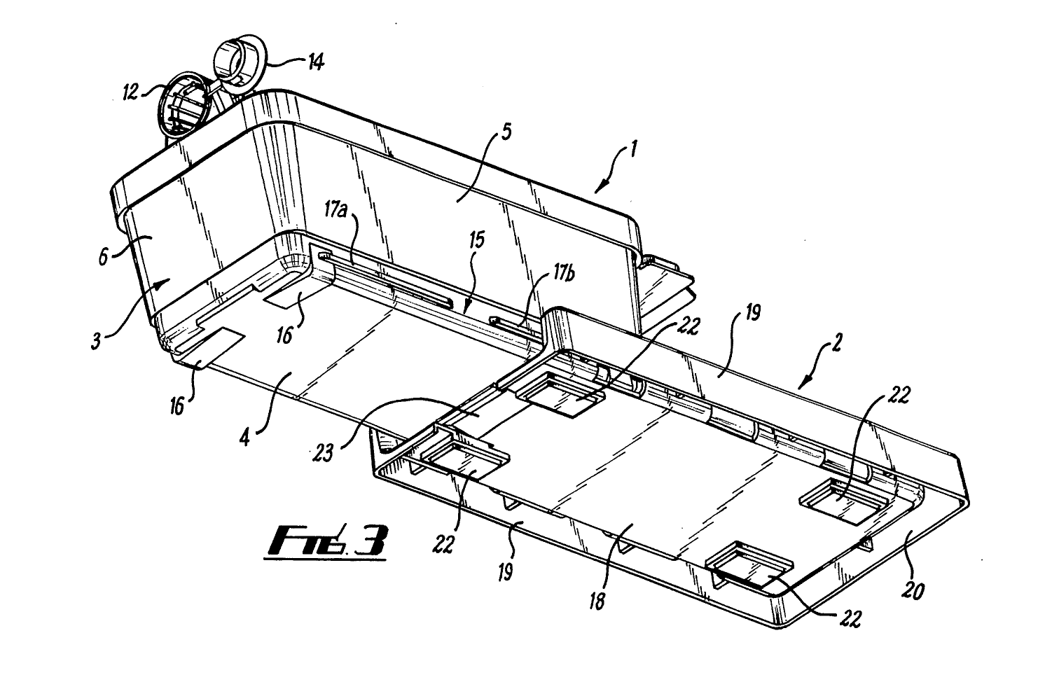

FIG. 3 is a bottom perspective view of the arrangement of FIG. 2.

Referring to the drawings these show a sharps container, for disposal of used drug cartridges and needles of a dental syringe, such container comprising a receptacle 1 and a base 2.

Specifically, the receptacle shown is adapted for use with a syringe (not shown), as described in WO 03/047664A, having a needle screwed onto a pull-off mounting device at the forward end of a syringe barrel which contains a disposable drug cartridge, a tubular sheath being mounted on the barrel for movement between a forward needle-sheathing position and a retracted needle-exposing position.

The receptacle 1 comprises an open-topped one-piece moulded plastics body 3 of generally rectangular horizontal cross-section having a generally flat horizontal bottom wall 4 and generally flat side and end walls 5, 6 which extend vertically or slightly inclined outwardly away from the bottom wall 4. The open top is bounded by a rim and is covered by an attached plastics lid 7.

The lid has a transverse slot-shaped opening 8 at a front end which can be sealed by a hinged snap-fit cover 9, and running from the opposite rear end up to the region of this opening 8 there is a longitudinally extending upstanding tunnel 10 which is open at the rear end and closed at its front end. The tunnel 10 is open to the interior of the receptacle 1 at its bottom periphery. Apart from the tunnel 10 and the opening 8 the lid 7 is solid i.e. with no other openings.

Within the tunnel 10 in communication with the open rear end there is a tube 11 extending part way along the tunnel with a peripheral collar 12 at the rear end and gripper fingers 13 at the front end. A cap 14 is provided for closing the rear end.

The aforesaid syringe with the needle sheathed can be inserted into the tube 11 so as that the sheath is pushed back by the collar 12 and the fingers 13 grip behind the needle mounting device whereby retraction of the syringe then causes the needle and mounting device to be pulled off so as to drop into the receptacle 1. The spent drug cartridge can then be dropped through the slot opening 8 into the receptacle 1.

Reference is made to WO 03/047664A for further details of the syringe and the mechanism within the tunnel 10 for removing the needle.

The bottom end portion 15 of the receptacle body 3, as shown, is stepped slightly inwardly around its entire periphery relative to the adjoining upper region of the body 3 so as to define a reduced dimension bottom end portion 15.

The bottom wall 4 of this end portion 15 is generally flat but at each corner has a slightly downwardly projecting foot 16, the four feet 16 being coplanar at their lower peripheries.

Along each of the two side walls 5, in the bottom end portion, there is a respective guide rail 17 defined by two spaced outwardly projecting aligned ridges 17a, 17b parallel to the bottom wall 4 and running along substantially the whole length of the receptacle 1.

The base 2 is in the form of a one-piece moulded plastics tray having a substantially flat rectangular horizontal bottom wall 18 bounded at its two long and one short edge by integral upstanding low common-height side and end walls 19, 20, thereby leaving the other short edge open. The height of these walls 19, 20 above the bottom wall is substantially equal to the height of the bottom end portion of the receptacle.

The length of the bottom wall 18 of the base 2 is slightly greater than the length of the bottom wall 4 of the receptacle 1.

The peripheral side wall 19 along each of the two long edges contains inwardly projecting short lips 21 longitudinally aligned and spaced apart in the central region of the respective inner surface of the wall 19. The aligned lips 21 along one side wall 19 are horizontally aligned with the aligned lips along the opposite side wall 19 and are parallel to the bottom wall 18.

The bottom wall 18 has at its under-surface at each corner a respective downwardly projecting rectangular foot 22, the four feet 22 having coplanar lower surfaces to which rectangular self adhesive pads are attached.

Adjacent the open short edge of the bottom wall 18, centrally of such edge, there is formed a resiliently deflectable rectangular tab 23 which is separated from the adjacent regions of the bottom wall 18 along its rear and longitudinally extending side edges i.e. so as to be attached only at its forward side edge. The tab 23 is formed with an upwardly directed wedge-shaped top surface 24 inclined upwardly forwardly.

The length of the bottom wall 18 between the forward upright end of this wedge shaped top surface 24 and the inner surface of the peripheral end wall 20 at the forward short edge is substantially equal to the length of the bottom wall 4 of the receptacle 1.

With the arrangement described above, in use the base 2 is fixed on a table top by means of the adhesive pads on the feet 22. The receptacle 1 is mounted on the base 2 by sliding along the channel defined between the peripheral side walls 19 at the long edges of the bottom wall 18. The receptacle 1 is inserted with the forward end, i.e. the end having the slot opening 8 thereat, first.

The bottom end portion 15 fits in the channel between the peripheral side walls 19 with the rails 17 slidably received beneath and in contact with the lips 21. The bottom wall 4 of the receptacle 1 bears on the wedge shaped surface 24 and deflects the tab 23 downwardly out of the way.

The receptacle 1 can be pushed in to a position at which the front end of the receptacle 1 abuts the peripheral end wall 20 at the short front edge, and the rear end of the receptacle 1 is now just clear of the wedge shaped surface 24 of the tab 23 whereby the tab 23 returns, under its own resilience to its normal upward position.

The receptacle 1 is thereby securely held longitudinally between the forward upright surface of the wedge shaped surface 24 of the tab 23 and the peripheral end wall 20 at the short front edge. It is also securely held against vertical separation from the base 2 by interengagement of the rails 17 and lips 21.

The receptacle 1 is thereby securely held relative to the table top. It cannot readily be displaced from the table, and the syringe can be pushed into the tube 11 and retracted therefrom without requiring the receptacle 1 to be held manually. Safety and in particular avoidance of needle stick injuries can thereby be avoided.

When required the receptacle 1 can be readily detached from the base 2 by manually deflecting the tab 23 downwardly and then sliding the receptacle 1 rearwardly out of the base 2.

When out of the base 2 the receptacle 1 can stand on the table top on the feet 16 in a stable manner.

With the arrangement described above, secure retention of the receptacle 1 can be ensured with a robust versatile easy to manufacture construction.

It is of course to be understood that the invention is not intended to be restricted to the details of the above embodiment, which are described by way of example only.

In particular, it is to be understood that the invention is not restricted to use with the specific kind of syringe described nor even to sharps disposal. The invention may be applied to the disposal of any suitable kind of sharps or non-sharps unsafe waste and depending on the use, the waste inlet or inlets to the receptacle will be appropriately constructed.

Also it is to be understood that the invention is not restricted to table top mounting. Alternatively or additionally the base may be appropriately adapted for any suitable mounting whether on a level table top surface or an upright wall or otherwise. Thus, the base may have a side bracket for wall mounting. It is also to be understood that the base may be provided with two or more fixings e.g. bottom adhesive pads as well as a wall bracket so that the user can select which fixing means to use.

1. A container for disposal of unsafe waste comprising a receptacle with an inlet thereto for waste and a mounting device for the receptacle in the form of a base which can be attached to a surface, the receptacle being interconnectable with the base through cooperable configurations wherein the cooperable configurations comprise respectively a channel and a projecting part, said channel and part being interconnectable by longitudinal sliding of the part along the channel, and at least one retention device being provided to restrict the extent of said sliding in at least one longitudinal direction.

2. A container according to claim 1, wherein the inlet of the receptacle of the container has a gripper or abutment device for engagement with a pull off hub of a syringe needle or a pull off mounting device on which a syringe needle is mounted.

3. A container according to claim 2, wherein deflectable gripper fingers are provided at the leading end of a tubular syringe-receiving said inlet.

4. A container according to claim 1, wherein the receptacle is a bin or box having closed or closable side walls, a bottom wall, and a top wall.

5. A container according to claim 4, wherein the inlet is in the top wall and the top wall is defined by a separate lid structure.

6. A container according to claim 1, wherein the body of the receptacle is of elongate rectangular cross-section, with its long axis in the aforesaid longitudinal direction of the channel.

7. A container according to claim 1, wherein the base is of larger cross-section, in all transverse directions, than the receptacle.

8. A container according to claim 1, wherein the base has a flat underside or at least coplanar parts for positioning on a level support surface.

9. A container according to claim 1, wherein the base is provided with an upwardly directed side extension for wall mounting.

10. A container according to claim 1, wherein the base is fixed relative to a surface by means of one or more adhesive tabs between the base and the surface.

11. A container according to claim 1, wherein the channel is provided in the base and the projecting part is provided on the receptacle.

12. A container according to claim 11, wherein the receptacle is of elongate rectangular cross-section, and the projecting part is defined by a lowermost portion of the receptacle.

13. A container according to claim 11, wherein the projecting part extends along substantially all of the length of the receptacle, and the channel is at least substantially as long as the projecting part.

14. A container according to claim 1, wherein the retention device is operable to restrict the extent of sliding along the channel in both directions.

15. A container according to claim 14, wherein the retention device comprises a closure or abutment at one end of the channel and a catch member at the opposite end of the channel, the catch member being spring urged or otherwise arranged so as to deflect to allow sliding of the projecting part passed the catch member into the channel and then to return to a locking position at which it obstructs return sliding of the projecting part.

16. A container according to claim 15, wherein the retention device ensures the receptacle does not detach from the base when a force is applied to the receptacle in a longitudinal direction on withdrawal of an associated implement from the inlet.

17. A container according to claim 14, wherein a further retention device is provided to restrain separation of the projecting part out of the channel transversally to the sliding direction.
